(19) United States
(12) Patent Application Publication
(10) Pub. No.: US 2013/0089592 A1
(43) Pub. Date: Apr. 11, 2013

(54) METHOD OF PRODUCING A HYDROGEL

(71) Applicants: Willem Heber Van Zyl, Stellenbosch (ZA); Annie Fabian Abel Chimphango, Pinelands (ZA); Johann Ferdinand Gorgens, Stellenbosch (ZA)

(72) Inventors: Willem Heber Van Zyl, Stellenbosch (ZA); Annie Fabian Abel Chimphango, Pinelands (ZA); Johann Ferdinand Gorgens, Stellenbosch (ZA)

(73) Assignee: STELLENBOSCH UNIVERSITY, Stellenbosch (ZA)

(21) Appl. No.: 13/703,228

(22) PCT Filed: Jun. 7, 2011
(86) PCT No.: PCT/IB2011/001245

§ 371 Date: Dec. 10, 2012

(30) Foreign Application Priority Data
Jun. 8, 2010 (ZA) 2010/03274

(51) Int. Cl. A61K 47/36; A61K 9/48; C12P 19/04; A61K 38/44

(52) U.S. Cl. CPC A61K 47/36; A61K 38/44; A61K 9/4833; C12P 19/04; Y10S 977/906; B82Y 5/00
USPC 424/408; 514/777; 424/400; 424/451; 514/1.1; 424/94.1; 424/405; 424/490; 424/419; 435/101; 977/906; 977/898; 977/795

(57) ABSTRACT

The invention provides a method of enzymatically modifying xylan by selectively removing glucuronic acid and/or arabinose side chains from the xylan with a-D-glucuronidase and/or a-L-arabinofuranosidase, and allowing the modified xylan to form into a hydrogel when the xylan becomes insoluble. A bioactive substance, such as a protein, enzyme, antimicrobial agent, bactericide or pharmaceutical compound can be added to the xylan so that the substance is encapsulated within the hydrogel or incorporated onto its surface. The hydrogel can be used as a drug delivery agent, such as for sustained-release or targeted drug delivery, rectal drug delivery or a dressing for a wound, burn or scar. The hydrogel can also be used as a coating, such as on medical gloves, catheters, surgical drainage systems, utensils or the like, or can be used in a scaffold for tissue engineering.

### FIELD OF THE INVENTION

This invention relates to a method of producing a hydrogel and to the hydrogel composition produced by the method.

### BACKGROUND TO THE INVENTION

Hydrogels are useful in the biomedical, pharmaceutical, agricultural, water treatment, nutriceutical and bioremediation industries, as well as in the production of functional packaging, sanitary and textile products. Hydrogels have specific application in the immobilisation and/or encapsulation of bioactive substances, such as for the preparation of slow delivery systems to facilitate long term delivery of an encapsulated substance.

Current sources of biodegradable biopolymers for the production of nano and micro hydrogels include β-(1-4) linked polysaccharides present in cellulose and starch [Ebringerová and Heinze, 2000, Macromol. Rapid Comm 21:542-556; Dumitriu, 2002, Polymeric biomaterials pp 133-147. Marcel Dekker Inc. New York], due to their high molecular mass and relatively lower degree or absence of substitution. However, starch and cellulose have existing competing uses as food additives and source of sugars for biofuels production. Alternative and relatively inexpensive sources of biodegradation polymers are therefore in demand.

Hydrogels can be chemically produced from aqueous extracts of various types of hemicelluloses by treatment with specific oxidating reagents. One of the major hemicelluloses in most plant cell walls is xylan, which is a soluble polymer in water. About 1.25 million tons of xylan are estimated to go to waste in South Africa every year as a by-product of the kraft pulping industry.

Depending on its source, xylan contains a backbone chain of 1,4-β-linked D-xylose residues to which L-arabinose, D-glucuronic acid, short oligosaccharide side chains, O-acetyl groups and feruloyl and p-coumaroyl are attached. Hydrogel formation is often achieved through processes such as hydrothermal treatment, subjection to supercritical and compressed fluids, and manipulation of pH ionic charges as in coacevation process [Gabrielii et al., 2000, Carbohydr. Polym. 43:367-374; Garcia et al., 2001, Polym. Bull. 46: 371-379; Coviello et al., 2007, J. Controll. Rel. 119:5-24]. These processes result in a gel formed during the intra- and/or intermolecular crosslinking of arabinoxylan and proteins. Only a few oxidating reagents have been reported to cause gel formation in biopolymers and these include hydrogen peroxide, usually in conjunction with peroxidase, ammonium peroxosulfate and formamidine disulfate. Hydrogel formation of hemicellulose may also be carried out by the addition of further biopolymers, e.g. of different quantities of chitosan [Gabrielii et al., 2000, Carbohydrate Polymers 43, 367-374]. Gel formation is then effected in a strongly acidic medium at 95° C.

Such crosslinking methods, however, produce hydrogels that are incompatible for direct use in pharmaceutical applications (in some cases it is possible to further process the chemically-formed hydrogels to remove chemicals residues which are not suitable for pharmaceutical use, but these steps are time-consuming an expensive). Furthermore, methods such as precipitation, polyelectrolyte complexation and desolvation [Garcia et al., 2001, Polym. Bull. 46: 371-379; Wang et al., 2006, J. Mater. Chem. 16:3252-3267; Silva et al., 2007, Int. J. Pharm. 334:42-47; Daus and Heinze, 2009, Macromol. Biosci. 9 DOI: 10.1002/mabi.200900201] for the preparation of nanohydrogels from polysaccharides are complex and costly. Not only are these thermal, physical and chemical methods inefficient due to the non-specific removal of side chains, but exposure to increased temperatures can result in the denaturing of thermolabile bioactive substances to be encapsulated, and the addition of chemicals cause downstream contamination of the resultant hydrogel with residual chemicals. Many of these slow delivery hydrogels also become prematurely degraded, before delivery of the encapsulated substance to the target location has been achieved. It is therefore a technical challenge to achieve adequate encapsulation of thermolabile bioactive substances, such as highly soluble proteins, within nano-sized chemostable hydrogels.

Accordingly, there exists a need for an alternative method of forming a hydrogel composition, especially for use in the encapsulation of thermolabile bioactive substances for drug delivery applications.

### SUMMARY OF THE INVENTION

A method of producing a hydrogel, the method comprising the steps of:
enzymatically modifying xylan which contains glucuronic acid (or a derivative, such as methyl glucuronic acid) and/or arabinose side chains so that it has reduced solubility in water compared to naturally occurring xylan, by selectively removing glucuronic acid and/or arabinose side chains from the xylan with one or both of α-D-glucuronidase and α-L-arabinofuranosidase; and
allowing the modified xylan to form a hydrogel.

The method may further include the step of contacting the modified xylan with a bioactive substance and allowing the bioactive substance to be immobilized on or within the hydrogel when it forms. The bioactive substance may be added to the xylan before or after it is modified, or before or after the hydrogel is formed. The bioactive substance may be a protein, enzyme, antimicrobial agent, bactericide, pharmaceutical compound or the like. A dispersion step, such as sonication, may be performed to disperse the bioactive substance within the hydrogel.

The xylan may be modified by selectively removing only glucuronic acid side chains with α-D-glucuronidase, by selectively removing only arabinose side chains with α-L-arabinofuranosidase, or by selectively removing both glucuronic acid and arabinose side chains with α-D-glucuronidase and a-L-arabinofuranosidase. Acetyl groups may also be selectively removed from the xylan with acetyl xylan esterase.

The α-L-arabinofuranosidase may be recombinantly produced.

The xylan modification step is preferably carried out without the main xylan chain being damaged or degraded, and more prefereably in the substantial absence of hydrolytic enzymes other than the ones used to remove the xylan side chains, and in particular in the absence of xylanase.

The xylan source may be sugarcane bagasse, bamboo, eucalyptus, pine, oatspelt and birch.

The method may further include a step of extracting the xylan from a lignocellulosic material prior to the modification of the xylan.

A sufficient number of side chains may be removed from the xylan by the enzyme so as to reduce the water solubility of the modified xylan.

The xylan may be modified at a temperature of from about 30° C. to about 60° C., and more preferably at from about 35° C. to about 45° C. or at from about 40° C. to about 50° C. The extracted xylan may be contacted with the enzyme for between about 9 and about 72 hours or between about 15 and 28 hours.

The pH may be from about pH 4 to about pH 6.

The extracted xylan to enzyme ratio may be about 5:2, and the xylan loading may be from about 12.5 mL.g⁻¹ to about 25 mL.g⁻¹. The α-L-arabinofuranosidase enzyme loading may be about 2.5-5 mL.g⁻¹ and the α-L-arabinofuranosidase may have a volumetric activity of about 18 nKat mL⁻¹. The α-D-glucuronidase enzyme loading may be about 0.2 mL.g⁻¹ and the α-D-glucuronidase may have a specific activity of about 300 nKat mg⁻¹. The concentration of xylan may be from about 0.32% to about 3.68%.

According to a second embodiment of the invention, there is provided a hydrogel formed by the method described above.

The hydrogel may be formed from micro- or nano-particles of from about 18 nm to about 1.48 μm, and preferably less than about 100 nm. The particles may be substantially spherical and may have a surface charge (zeta potential) in the range of from about −19 to about −1.03 mV.

The hydrogel may include a bioactive substance or molecule, such as a protein, enzyme, antimicrobial agent, bactericide or pharmaceutical compound.

The bioactive substance or molecule may be encapsulated within the hydrogel composition or may be immobilized on the surface of the hydrogel.

The hydrogel composition may be for use in drug delivery, such as sustained-release or targeted drug delivery, or as a dressing for a wound, burn or scar. The hydrogel composition may also be for use as a coating or in a scaffold for tissue engineering.

### BRIEF DESCRIPTION OF THE FIGURES

One embodiment of the invention will be described, by way of example only, with reference to the drawings in which:

FIG. 1 shows a schematic representation of plasmid pGTP-AbfB: the abfB gene was cloned into the NotI site of pGTP, with the transcriptional control of abfB directed by the glyceraldehyde-3-phosphate dehydrogenase promoter (gpdp) of A. nidulans and glucoamylase terminator (glaA_{T}) of A. awamori;

FIG. 2 is a graph showing the production characteristics of AbfB by recombinant A. niger D15[abfB] in shake flasks in pellet form;

FIG. 3 is a graph showing the production characteristics of AbfB by recombinant A. niger D15[abfB] in shake flasks produced in 2×MM media (std), 1% concentrate corn steep liquor enriched media (CCSL1), 2% concentrate corn steep liquor enriched media (CCSL2) and 10% concentrate corn steep liquor enriched media (CCSL10), vertical bars denote 0.95 confidence intervals;

FIG. 4 is a graph showing the production of AbfB in 10 L BIOFLO 110 bioreactor (mycelial morphology) indicating release of extracellular AbfB activity;

FIG. 5 shows the mycelial morphological changes of A. niger D15[abfB] at different time intervals during cultivation;

FIG. 6 shows graphs of the relative enzyme activity (%) of recombinant AbfB produced by A. niger D15[abfB] over a range of (A) pH and (B) temperature using p-NPA as substrate (vertical bars denote standard deviation);

FIG. 7 shows graphs of the (A) optical density at 405 nm of a A. niger D15[abfB] culture over time at various pH, and (B) the relative enzyme activity (%) over time at various temperatures. AbfB assays were conducted under standard conditions (vertical bars denote 0.95 confidence intervals, p<0.01);

FIG. 8 shows a graph of the remaining relative activity (%) of the recombinant AbfB after storage at 26, 30 and 37° C.;

FIG. 9 shows a graph of the enzyme activity of AbfB in the presence of varying concentrations of pNPA substrate, demonstrating the effect of p-NPA concentration on reaction rate by AbfB produced in (a) bioreactor on CCSL enriched medium and (b) in shake flasks on CCSL enriched medium. Dotted lines show values derived for Vmax and Km;

FIG. 10 shows a graph of the enzyme activity of AbfB in the presence of varying concentrations of pNPA substrate, demonstrating the effect of p-NPA concentration on reaction rate by (a) partially purified AbfB produced in a bioreactor on enriched medium and (b) by AbfB produced in standard 2×MM medium. Dotted lines show values derived for Vmax and Km;

FIG. 11 is a silver stained (Biorad) 10% Sodium dodecyl sulfate-polyacrylamide electrophoresis (SDS-Page) gel on which the crude AbfB preparation was resolved. The crude AbfB preparation was produced by recombinant A. niger cultivated on 2×MM medium in shake flasks (Lanes (a) bench mark pre-stained protein marker (Invitrogen Cat no 10748010); b AbfB negative control; (c) and (e) AbfB supernatant diluted 10×; and (d) and (f) AbfB supernatant 5× diluted;

FIG. 12 is a comparison of silver stained (Biorad) 10% SDS-Page of crude AbfB lane that is (a) produced in CCSL enriched medium in a 10 L bioreactor diluted 20×; (b) CCSL enriched medium in bioreactor at 10× dilution; (c) in standard medium in shake flask at 20× dilution; (d) in standard medium in shake flask at 10× dilution; (e) AkTA partially purified AbfB produced in CCSL enriched medium in fermenters at 20× dilution; and f) AkTA partially purified AbfB produced in CCSL enriched medium in fermenters at 10× dilution AbfB produced in standard 2×MM medium;

FIG. 13 shows particle size distribution by volume statistics of (a) unmodified oatspelt xylan; (b) AbfB treated oatspelt xylan hydrogel after 30 min; (c) unmodified oatspelt xylan after 3 h; (d) AbfB treated oatspelt xylan gel after 3 h; (e) unmodified xylan after 7 days; and (f) AbfB treated oatspelt xylan hydrogel after 7 days;

FIG. 14 shows response surface plots showing effect on (a) particle size and (b) zeta potential of (i) AbfB hydrolysis time and xylan concentration at constant PEG1000 concentration of 27.5%; (ii) PEG1000 concentration and xylan concentration at constant of time of 15.5 h; and (iii) of PEG1000 and time at constant xylan concentration of 2%. The response surface plots for particle size fitted the quadratic model with R²=0.88 and adjusted: R²=0.81 and for Zeta potential fitted with R²=0.74 and adjusted: R²=0.56. Significance of the effects was calculated p<0.05;

FIG. 15 shows desirability surface plots of the effect of xylan concentration, PEG1000 and AbfB hydrolysis duration on (a) particle size and (b) zeta potential. Plot in (i) shows interaction between time and xylan concentration; in (ii) PEG1000 concentration and xylan concentration; in (iii) PEG1000 and time; and in (iv) size and significance of the main effects and interactions. Effect estimates for particle size gave R²=0.88; Adjusted R²=0.81, MS Residual=4228775, and effect estimates for Zeta potential gave R²=0.73536; Adjusted R²=0.56 MS Residual=49.29429;

FIG. 16 shows encapsulation and release of HRP, (a) residual activity in reaction mixture where HRP was added before (AHB) and after (AHA) in AbfB formed oatspelt xylan hydrogels at 50 and 100 μL. The HX denotes residual activity of HRP in untreated xylan; (b) diffusion progression of HRP out of oatspelt xylan gel when added before AbfB treatment (AHB), after AbfB (AHA), the line graph represents stability of HRP+ve control (H) during the assay period; and (c) the rate of diffusion for HRP out of AbfB modified oatspelt xylan gel with HRP added before formation of xylan hydrogels (AHB) and after formation of xylan hydrogels (AHA);

FIG. 17 shows a hydrogel formed after removal of MeGlcA from birch xylan by AguA at 40v° C., pH 4.8 for 24 h at xylan specific dosage of purified α-D-glucuronidase of 8000, 5000 and 2500 nkat g⁻¹ substrate; and

FIG. 18 shows the amount of MeGlcA removed from birch xylan by AguA at 40v° C., pH 4.8 for 24 h at xylan specific dosage of purified α-D-glucuronidase of 8000, 5000 and 2500 nkat g⁻¹ substrate.

### DETAILED DESCRIPTION OF THE INVENTION

A method of producing a hydrogel and the hydrogel produced thereby are described herein. The applicant has found that by enzymatically removing glucuronic acid and/or arabinose side chains from water-soluble xylan containing these side chains, without degrading or damaging the main xylan chain, it is possible to form a hydrogel from the modified xylan. The enzyme which is used to selectively remove the glucuronic acid and/or arabinose side chains is an α-L-arabinofuranosidase (AbfB) (EC 3.2.1.55) and/or an α-D-glucuronidase (AguA) (EC 3.2.1.131-). If the xylan also contains acetyl side chains, then an acetyl xylan esterase (EC 3.1.1.72) can also be used to selectively remove these groups. A bioactive substance is added to the modified (desubstituted) xylan, which is insoluble in water, so that the bioactive substance is incorporated onto or into the hydrogel, either during formation of the hydrogel or after the formation thereof.

The xylan can be from any source with water-soluble xylan having glucuronic acid and/or arabinose groups, such as sugarcane bagasse, bamboo, eucalyptus, pine, oatspelt or birch.

Examples of suitable bioactive substances include proteins, enzymes, antimicrobial agents, bactericides, pharmaceutical compounds and the like. A dispersion step, such as sonication, can be performed to disperse the bioactive substance within the hydrogel.

Hydrogels formed by this method comprise particles which are nano- or micro-sized, allowing the hydrogel to be used as a drug delivery agent, such as for sustained-release or targeted drug delivery (e.g. for cancer drugs), rectal drug delivery or a dressing for a wound, burn or scar. The hydrogel can also be used as a coating, such as on medical gloves, catheters or surgical drainage systems, in a scaffold for tissue engineering, or even as a coating where the bioactive substance entrapped on or within the hydrogel modifies the surface characteristics of the substrate on which the hydrogel is coated, for example, a bactericidal coating on a cellulosic surface, such as a cardboard plate.

The α-L-arabinofuranosidase (EC 3.2.1.55), α-D-glucuronidase (EC 3.2.1.131-) or acetyl xylan esterase (EC 3.1.1.72), either alone or in combination, can be used to remove the xylan side chains. These enzymes should be purified or recombinantly produced so as to ensure that they are not contaminated with other hydrolytic enzymes which might degrade or damage the main xylan chain, such as xylanase. A sufficient number of side chains should be removed by the enzyme so as to reduce the water solubility of the modified xylan and allow it to form a hydrogel.

The xylan is modified at a temperature of from about 30° C. to about 60° C., and more preferably from about 35° C. to about 45° C. or from about 40° C. to about 50° C. The xylan can be contacted with the enzyme for at least about 30 minutes, but more preferably between about 9 and about 72 hours, or between about 15 and about 28 hours. The pH can be from about pH 4 to about pH 6. A plasticizer may also be added to the xylan and enzyme composition to form the hydrogel.

The extracted xylan to enzyme ratio is generally about 5:2, and the xylan loading is from about 12.5 mL.g⁻¹ to about 25 mL.g⁻¹. The α-L-arabinofuranosidase enzyme loading can be about 2.5-5 mL.g⁻¹ and the α-L-arabinofuranosidase can have a volumetric activity of about 18 nKat mL⁻¹. The α-D-glucuronidase enzyme loading can be about 0.2 mL.g⁻¹ and the α-D-glucuronidase can have a specific activity of about 300 nKat mg⁻¹. The concentration of xylan is generally from about 0.32% to about 3.68%.

The hydrogel composition is generally composed of micro- or nano-particles having an average size of from about 18 nm to about 1.48 μm, such as less than about 100 nm. The particles can be substantially spherical and can have a surface charge (zeta potential) in the range of from about −19 to about −1.03 mV.

In the examples below, recombinant α-L-arabinofuranosidase (AbfB) and purified α-D-glucuronidase (AguA), enzymse with ability to specifically remove nonreducing α-L-arabinofuranoside and glucuronic acid side chains, respectively, from polymeric xylan, were used to selectively remove the arabinose and glucuronic acid side groups from oatspelt and birch xylan leading to formation of hydrogels. The hydrogels were characterized for particle size, surface charge (zeta potential) and ability to encapsulate bioactive agents for slow release delivery. The xylan hydrogels produced by the AbfB had particle sizes ranging from 18 nm to >10,000 nm. The hydrogels were spherical and exhibited negative zeta potential of up to −19 mV. The morphological characteristics and stability of the xylan hydrogels were negatively influenced by xylan concentration and AbfB reaction time. The xylan hydrogels encapsulated horse radish peroxidase (HRP) as an example of a bioactive substance and slowly released it over a period of 180 min while still in active form.

AbfB thus presents a novel tool for transforming soluble xylan into functional nanohydrogels for use as encapsulation matrices and slow delivery system for soluble bioactive substances.

Further features of the invention will now become apparent from the following non-limiting examples with reference to the accompanying figures. Although only horse radish peroxidase is used as an example of a bioactive agent which can be encapsulated or entrapped within the hydrogel, it will be apparent to a person skilled in the art that other substances could also be used. It will also be apparent that α-L-arabinofuranosidase and α-D-glucuronidase are able to selectively remove arabinose and glucuronic acid side chains from other sources of xylan, and the disclosures in co-pending PCT application entitled “Modification of Xylan” are incorporated herein.

### Examples

### Materials and Methods

### Materials

Oatspelt xylan solutions were prepared according to De Wet et al. [2008, Appl Microbiol Biotechnol 77:975-983] from oatspelt xylan (X-0627) purchased from Sigma. The oatspelt xylan contained 10:15:75 arabinose:glucose: xylose. The enzyme used was crude α-L-arabinofuranosidase (AbfB) with volumetric activity of 18.0 nKat mL⁻¹ on p-Nitrophenyl Arabinofuranoside (p-NPA). The enzyme was produced in-house in a fermenter at the Department of Microbiology of Stellenbosch University using a recombinant Aspergillus niger D15 microbial system as described below. The encapsulant bioactive agent used was horse radish peroxidase (EC 1.11.1.7) [HRP, Type VI-A P-6782 with specific activity of 1000 U mg⁻¹ against 2,2′-Azino-bis(3-ethylbenzthiazoline-6-sulfonic Acid) (ABTS) Sigma #A-1888)]. The H₂O₂ for use in the HRP assay was prepared from a 30% (w/w) stock solution purchased from Merck. The polyethylene glycol 1000 (PEG1000, #81190) used as plasticizer was purchased from Merck. The buffers were prepared from analytical grade chemicals according to Gomori [1955, Method. Enzymol. 1: 138-146]. All water used in the study was de-ionised water unless specified otherwise.

### Production of Recombinant α-L-Arabinofuranosidase (AbfB)

A recombinant α-L-arabinofuranosidase (AbfB) was produced by cloning the AbfB gene from A. niger into the protease deficient and medium non-acidifying strain A. niger D15, under the transcriptional control of the glyceraldehyde-3-phosphate dehydrogenase promoter (gpdp) of A. nidulans and the glucoamylase terminator (glaA_{T}) of Aspergillus awamori (A. awamori). The growth characteristics of the A. niger D15[AnabfB] strain and production levels of AbfB were studied in shake flasks and a bioreactor using defined standard media (2×MM media) and cornsteep liquor enriched media. The protein profiles, substrate specificity, substrate dependency, optimal pH and temperature, stability in application and storage, and recyclability were assessed as described below.

### Cultivation of Bacterial and Fungal Strains

The genotypes of the bacterial and fungal strains as well as the plasmids used are summarised in Table 1. Recombinant plasmids were constructed and amplified in E. coli DH5α. E. coli was cultivated at 37° C. in LB medium (1% yeast extract, 1% tryptone and 0.5% NaCl) on a rotary shaker at 100 rpm, supplemented with 100 μg/L ampicillin. The A. niger fungal strains were maintained at 30° C. in minimal media (MM) and on spore plates according to the procedure of Rose and Van Zyl [2002 Appl. Microbiol. Biotechnol. 58: 461-468]. The media in which the A. niger D15 parental strain was maintained was supplemented with 0.01 M uridine. Transformants were prepared according to the procedure of Rose and Van Zyl [2002 Appl. Microbiol. Biotechnol. 58: 461-468] and selected on MM lacking casamino acids and uridine. Transformants were cultivated in Erlenmeyer shake flasks (125 mL) containing 30 mL of double strength traditional minimal medium (2×MM) containing 10% glucose. The medium was inoculated to a final spore concentration of 1×10⁶ spores ml⁻¹. The A. niger strains were cultivated at 30° C. on a rotary shaker (New Brunswick Scientific, Edison, W. J., and U.S.A) at 120 rpm.

TABLE 1
Summary of the genotype and sources of the strains and plasmids used in
transforming Aspergillus niger

| | Genotype | Source |
|---|---|---|
| Strain: | | |
| A. niger D15 | pyrG prtT phmA (nonacidifying) | Wiebe et al., 2001, Biotechnol. Bioeng. |
| | | 76: 164-174 ATCC 9029 |
| A. niger van Tieghem | Wild-type | ATCC 10864 |
| A. niger D15[pGTP]* | A. niger D15 with gpdP-glaAT | Rose and Van Zyl, 2008, The Open |
| | | Biotechnol. J. 2: 167-175 |
| A. niger D15[abfB]* | A. niger D15 with gpdP-abfB-glaAT | This study |
| E. coli DH5α | supE44 ΔlacU169 (ø/80lacZΔM15) | Sambrook et al., 1989, Molecular |
| | hsdR17 recA1 endA1 gyrA96 thi-1 relA1 | cloning: a laboratory manual. Cold |
| | | Spring Harbor Laboratory, Cold Spring |
| | | Harbor, NY |
| Plasmids: | | |
| pBS-pyrGamdS | bla pyrG amdS | Plüddemann and Van Zyl, 2003, Curr. |
| | | Genet. 43: 439-446 |
| pGTP | bla gpdP-glaAT pyrG | Rose and Van Zyl, 2008, The Open |
| | | Biotechnol. J. 2: 167-175 |
| pGTP-abfB | bla gpdP-abfB-glaAT pyrG | This study |

*plasmids were integrated

Spores produced from A. niger D15 [abfB] plates were re-inoculated on rice (Tastic™ rice) sporulation medium to produce spores at a relatively higher concentration (>1×10⁶). To prepare the rice sporulation medium, tastic rice (20 g) was placed in Erlenmeyer shake flasks (250 mL) to which 8 mL of 0.1% (w/v) urea was added. The rice was then autoclaved at 121° C. for 15 min. Spores were added to each flask and incubated at 30° C. for a period of 3-6 days or until 80% of the rice surface was observed to be covered with spores. The spores were harvested into a 0.9% NaCl solution (approximately 50 mL for each flask) and stored in Schott bottles at 4° C.

A. niger spores were inoculated in 2×MM medium enriched with concentrated corn steep liquor (CCSL) which was prepared according to a modified version of the protocol of Gurlal et al. [2006, CSIR/B10/IR/PPD/2006/0023/B, CSIR, pp 1-8]. The CCSL was donated by Mr. Hough Joubert of African Products-Belleville, South Africa. The CCSL with initial pH of pH 3.87 was sterilised (121° C., 15 min, 1 bar) and filtered (0.22 μm pore size) before being added to the standard medium at 1%, 2%, and 10% (w/v). A. niger was cultivated in the respective media under standard A. niger cultivation conditions. Culture samples were taken at 24 h intervals for 7 days and the AbfB activity determined. Unless otherwise stated, the CCSL optimised medium was used in the subsequent cultivations of A. niger.

Cloning of pGTP and pGTP-AbfB Plasmid Constructs

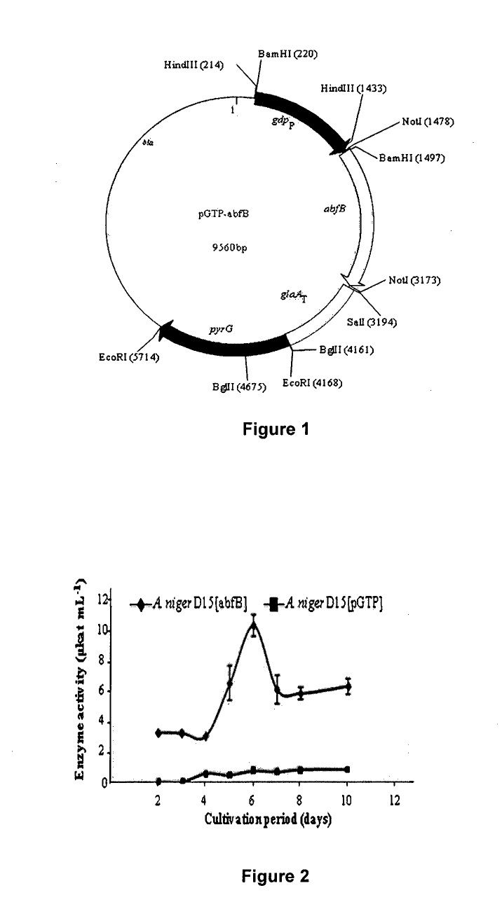

Standard protocols such as those described by Sambrook et al. [1989, Molecular cloning: a laboratory manual. Cold Spring Harbor Laboratory, Cold Spring Harbor, N.Y.] were followed for all DNA manipulations. The method used to construct the plasmids and the fungal strains was substantially according to that described by Rose and Van Zyl [2002 Appl. Microbiol. Biotechnol. 58: 461-468]. Briefly, the pyrG gene was amplified from pBS-pyrGamdS [Plüddemann and Van Zyl, 2003 Curr. Genet. 43: 439-446] by standard PCR techniques and cloned into the EcoRI site of plasmid pGT [Rose and Van Zyl, 2002, Appl. Microbiol. Biotechnol. 58: 461-468] to generate pGTP. For the production of recombinant α-L-arabinofuranosidase (AbfB), the abfB gene was amplified from the genome of A. niger 10864 using standard PCR methods. The abfB was then cloned into the NotI site of pGTP, generating pGTP-AbfB (FIG. 1) in which the glyceraldehyde-3-phosphate dehydrogenase promoter (gpd_{P}) of A. nidulans and the glucoamylase terminator (glaA_{T}) of A. awamori were located upsteam and downstream of the abfB gene respectively. Plasmids pGTP and pGTP-AbfB were then integrated into the genome of A. niger D15 in multiple sites according to standard techniques. A total of 100 putative A. niger D15[abfB] transformants were prepared and screened for extracellular AbfB activity. The selected A. niger D15[abfB] transformants were then cultivated in shake flasks in at least three replicates (with 3 repetitions) to evaluate stability in cultivation conditions with respect to extracellular AbfB production.

Cultivation of A. niger D15[abfB]

The morphological and growth characteristics of A. niger D15[abfB] were monitored visually and by electronic microscopy. Mycelia and pellets from bioreactor and terminated shake flasks cultures were observed under a microscope (100× magnification) prior to filtering through Mira cloth mesh. The biomass was washed with deionised water (dH₂O) before being transferred into pre-weighed aluminum foils for drying in an oven at 60° C. until a constant weight was obtained. The biomass concentration was calculated as an average dry weight of biomass (dry wt) per unit volume of the culture (g L⁻¹).

The pellet formation was achieved by inoculating the medium (either 2×MM or CCSL (2% w/v) enriched 2×MM medium) with 1×10⁶ spores mL⁻¹ and incubating the shake flasks at 30° C. on a shaker (120 rpm). The cultivation medium had an initial pH of 5.5-6.0 and 5.0 for 2×MM and CCSL enriched, respectively. Mycelial production of AbfB by A. niger D15[abfB] was carried out in a 14 L capacity stirred tank bioreactor (BIOFLO 110 modular bench top fermentation system, New Brunswick Scientific company, Inc, USA) containing 8 L of optimised medium enriched with CCSL (2% w/v) and a spore inoculum of 1×10⁶ spores mL⁻¹ maintained at 30° C. Mass transfer was achieved using a single 3 bladed pitched impeller (30×20 mm) at an agitation speed regulated between 350-700 rpm, depending on the level of the dissolved oxygen (DO) in the bioreactor. The DO was maintained above the critical point (>20%) with air and oxygen supplied through a sparger and the levels regulated by a rotameter at 0.5 vessel volume per min (0.5 VVM). Sampling was done through a port installed with a 0.2 μm air filter connected to a syringe for suction into JA 20 centrifuge bottles every hour for the initial 24 h and thereafter every 3-4 h for determination of extracellular AbfB activity, biomass growth and substrate concentration. Foaming was controlled by the addition of 0.1% (v/v) of antifoam A (30% aqueous emulsion with emulsifiers, A 5758, Sigma). Excess foam was collected in a foam trap. The cultures were terminated after 144 h.

### Fractionation and Partial Purification of AbfB Enzyme Preparations

The enzyme supernatant harvested from bioreactor fermentation cultures was filtered through Mira cloth. The filtrate was centrifuged (Beckman, J2-21 centrifuge) at 12 000 rpm for 10 min at 4° C. The resulting supernatant was filtered through 0.2 μm filters and concentrated using an Amicon system, Diaflo Ultrafilter PM 10 concentrator, (Amicon Division, W.R. Grace & Co., USA) or a Millpore Minitan ultrafiltration system (Millpore Corporation) with MWCO of 10 kDa. The choice of the ultrafiltration system was based on the initial sample volume. Concentration of crude enzyme samples was carried out by lyophilising the enzyme supernatant in Virtis freeze dryer. The concentrated enzyme supernatant was subjected to ammonium sulfate fractionation at a percentage saturation of 60 and 80% while mixing at 200-250 rpm for 4 h at 4° C. The protein mixture was centrifuged at 1200 rpm for 1 h at 4° C. The pellet obtained at the desired saturation level was re-suspended in 5 mL of Milli-Q water and dialysed against 2 L of 10 mM acetate buffer (pH 5.0) at 4° C. overnight. The desalted concentrated enzyme supernatant was subjected to a single step partial purification in a fast performance gel filtration chromatography (AKTA purifier system, Amersham Pharmacia Biotech) installed with UNICORN computer control system (version 3.2). The protein sample (0.3 mL) was applied to a Superdex 75 HR 10/30 size exclusion column. The protein was eluted with 0.05 M acetate buffer pH 4.0 containing 0.4 M NaCl at a flow rate of 0.5 mL min⁻¹. Fractions demonstrating AbfB activity were pooled and concentrated using the Amicon system before molecular and kinetic analysis.

### AbfB Characterisation

### Determination of AbfB Molecular Weight (MW)

Enzyme preparations were resolved by 10% sodium dodecyl sulfate-polyacrylamide gel electrophoresis (SDS-PAGE) according to the method of Laemmli [1970, Nat. 227: 680-685]. The resolved proteins were initially stained with Coomassie Brilliant Blue R250 and subsequently with silver stain (Bio-Rad kit). The molecular weight (MW) of the resolved protein was estimated from the gel using BenchMark™ prestained protein marker (Invitrogen and Prestained protein ladder (Fermentas). The theoretical Mw and isoelectric point (pI) of the AbfB was estimated from the original protein sequence (protein length of 499 bases) using DNAMAN sequence analysis computer software.

Determination of α-L-arabinofuranosidase Activity of AbfB using pNPA

α-L-arabinofuranosidase activity of AbfB (AbfB activity) was determined using a colorimetric assay in which the release of p-Nitrophenol from p-Nitrophenyl-a-arabinofuranoside (C₁₁H₁₃NO₇, pNPA, Sigma) was measured. A 100 μL reaction mixture was prepared which consisted of 25 μL 5 mM pNPA in 0.05 M citrate buffer (pH 5.0), 25 μL deionised water (dH₂O), 25 μL of appropriately diluted supernatant and 25 μL 0.05 M citrate buffer (pH 5.0). The reaction was incubated at 40° C. for 10 min and was terminated by the addition of 100 μL saturated sodium tetraborate (Na₂B₄O₇, Sigma-Aldrich). The mixture was diluted five times before measuring the absorbance at 405 nm wavelength using a spectrophotometer. The AbfB activity was determined from a standard curve using pNitrophenol (pNP, C₆H₅NO₃, Sigma) as standard under similar assay conditions. The AbfB activity was calculated as the amount of enzyme capable of releasing 1 nmole of pNP from the pNPA substrate per mL per second (nkat/mL, conversion factor: 1 Unit=16.67 nkat). Unless stated otherwise, all assays were conducted in triplicate. Commercial α-L-arabinofuranosidase from A. niger (E-AFASE, Megazyme) was used as a positive control.

### Determination of pH and Temperature Optima for AbfB Activity

Recombinant AbfB was characterised with respect to optimal pH and temperature under the assay conditions described above. The pH optimum and pH stability of AbfB was determined using pNPA desolved in Mcllvaine's buffer [McIlvaine, 1921, J. Biol. Chem. 49: 183-186]. Optimal temperature conditions for and temperature stability of the recombinant AbfB was determined using pNPA in 0.05 M citrate buffer at pH 5.0. The effect of substrate concentration on AbfB activity was then determined by using pNPA prepared at varying concentrations in 0.05 M citrate buffer pH 5.0. The assays were conducted under AbfB assay conditions as described above. The saturation kinetic properties, maximum activity (Vₘₐₓ) and Michaelis Menten constant (Km) of partially purified and crude AbfB, were estimated from the plot of release of pNP as a function of substrate concentration.

### Production and Characterization of Oatspelt Xylan Hydrogels

About 2.5 mL oatspelt xylan solution (1% w/v) was placed in test tubes into which 1 mL of recombinant AbfB with volumetric activity of 18 nKat mL⁻¹ was added. The volume of the reaction mixture was adjusted to 5 mL by addition of 0.05 M citrate buffer pH 5.0. The reaction was performed at 40° C. in a water bath for 16 h. A control sample consisting of 2.5 mL oatspelt xylan was incubated under similar conditions in the absence of AbfB. Precipitation of the oatspelt xylan was visually assessed by taking pictures and microscopic analysis. Stability of the formed xylan hydrogels was assessed by measuring the mean particle size distribution in the reaction mixture after 30 min, 3 h and 7 days of terminating the AbfB xylan hydrolysis process using Nano ZS90 Zetasizer (Malvern Instruments, 2004). The Nano ZS90 Zetasizer determines particle size by measuring Brownian motion of the particles in a sample using principle of Dynamic Light Scattering (DLS). The results were analysed using an inbuilt Malvern software programme (Malvern Instruments, 2004)

Experimental Design for Assessing Effect of Plasticizer, Xylan Concentration and, Enzyme Hydrolysis Duration on Size and Surface Charge of Enzymatically Produced Xylan Hydrogels

The effects of PEG1000 plasticizer, oatspelt xylan concentration, and AbfB hydrolysis duration on xylan hydrogel size and surface charge were evaluated using a standard three factor-two level cubic plus star surface central composite design (CCD). The design consisted of 16 runs of which 8 were cube points (n_{c}), 6 were star points (nₛ) and 2 were a combined of center points in the cube portion of the design and center points in the star portion of the design (n₀) (Statistica, 2008). The region of experimentation of the independent variables at low and high levels (−1 and 1) was for xylan concentration (X1) 1%≦X1≦3%, for AbfB reaction time (X2) 7 h≦X2≦24 h and for PEG plasticizer concentration (X3) 15%≦X3≦40% (Table 2). The central points for X1, X2 and X3 were respectively, 2%, 15.5 h and 27.5%. The low and high levels of the independent variables were coded using the following formulae:

X1=(C1-2)/1 (1)

X2=(C2-27.5)/12.5 (2)

X3=(C3-15.5)/8.5 (3)

where X and C denote coded and natural independent values, respectively and subscripts 1, 2 and 3 denote xylan concentration, plasticizer and time. The α for rotatability in coded form was 1.6818 which was calculated from the following formula: α=(n_{c})^{1/4}, where n_{c} stands for the number of cube points in the design (i.e. points in the factorial portion of the design) (Statistica, 2008). The natural ±a values were for oatspelt xylan 0.32 and 3.68%, plasticizer 6.48 and 48.52% and for time 1.2 and 29.8 h. The response surface plot was fitted with a second order polynomial as follows:

Z=β₀+β₁ₓ₁+β₁₁ₓ₁_{²}+β₂ₓ₂+β₂₂ₓ₂_{²}+β₃ₓ₃+β₃₃ₓ₃_{²}+∈ (4)

where:
Z=particle size (nm) or zeta potential (mV)
β₀₊β₁ . . . βₙ=linear regression coefficient
β₁₁ . . . βₙₙ=quadratic regression coefficient
β=error
X₁, X₂, X₃=xylan concentration, AbfB hydrolysis duration (h) and PEG1000 concentration, respectively.

The size of effect for the individual parameters and their interaction was determined by regression analysis. In addition, a desirability function was used to determine optimal set point for the parameters that would give either the smallest particle size or the most negative zeta potential.

TABLE 2
Summary of the central composite design for evaluating effect of polyethylene glycol 1000 (PEG 1000) on particle size and zeta potential
of α-L-arabinofuranosidase produced oatspelt xylan hydrogels

| | | | | | | | COMPOSITION OF REACTION MIXTURE | | | | | |
|---|---|---|---|---|---|---|---|---|---|---|---|---|
| | | | | NATURAL VALUES | | | | Volume | | | OUTPUT | |
| | | | | Xylan | | | | 0.1M | | | Estimated | |
| | | | | Concen- | | PEG | Volume | Citrate | Volume | AbfB | Mean | Zeta |
| Sample | CODED VARIABLES* | | | tration | Time | 1000 (% | Xylan | buffer pH | PEG 1000 | Enzyme | Particle | Potential |
| Identification | X1 | X2 | X3 | (% w/v) | (h) | xylan wt) | (uL) | 6.0 (uL) | Plasticizer (uL) | **(uL) | size (nm) | (Mv) |
| XEP_1 | −1 | −1 | −1 | 1.00 | 7.00 | 15.00 | 2500 | 1475 | 25 | 1000 | 81 | −13.0 |
| XEP_2 | −1 | −1 | 1 | 1.00 | 7.00 | 40.00 | 2500 | 1433 | 67 | 1000 | 21 | −13.2 |
| XEP_3 | −1 | 1 | −1 | 1.00 | 24.00 | 15.00 | 2500 | 1475 | 25 | 1000 | 103 | −10.4 |
| XEP_4 | −1 | 1 | 1 | 1.00 | 24.00 | 40.00 | 2500 | 1433 | 67 | 1000 | 235 | −19.1 |
| XEP_5 | 1 | −1 | −1 | 3.00 | 7.00 | 15.00 | 2500 | 1425 | 75 | 1000 | 10000 | −1.03 |
| XEP_6 | 1 | −1 | 1 | 3.00 | 7.00 | 40.00 | 2500 | 1300 | 200 | 1000 | 10000 | −2.35 |
| XEP_7 | 1 | 1 | −1 | 3.00 | 24.00 | 15.00 | 2500 | 1425 | 75 | 1000 | 10000 | −2.37 |
| XEP_8 | 1 | 1 | 1 | 3.00 | 24.00 | 40.00 | 2500 | 1300 | 200 | 1000 | 10000 | −9.16 |
| XEP_9 | −1.68179 | 0 | 0 | 0.32 | 15.50 | 27.50 | 2500 | 1485 | 15 | 1000 | 292 | −15.5 |
| XEP_10 | 1.68179 | 0 | 0 | 3.68 | 15.50 | 27.50 | 2500 | 1332 | 168 | 1000 | 10000 | −9.5 |
| XEP_11 | 0 | −1.68179 | 0 | 2.00 | 1.20 | 27.50 | 2500 | 1408 | 92 | 1000 | 4378 | −5.18 |
| XEP_12 | 0 | 1.68179 | 0 | 2.00 | 29.80 | 27.50 | 2500 | 1408 | 92 | 1000 | 74 | −8.41 |
| XEP_13 | 0 | 0 | −1.68179 | 2.00 | 15.50 | 6.48 | 2500 | 1478 | 22 | 1000 | 144 | −17.1 |
| XEP_14 | 0 | 0 | 1.68179 | 2.00 | 15.50 | 48.52 | 2500 | 1339 | 161 | 1000 | 126 | −10.8 |
| XEP_15 | 0 | 0 | 0 | 2.00 | 15.50 | 27.50 | 2500 | 1408 | 92 | 1000 | 114 | −12 |
| XEP_16 | 0 | 0 | 0 | 2.00 | 15.50 | 27.50 | 2500 | 1408 | 92 | 1000 | 114 | −12 |

*Three factor standard two cubic plus star surface central composite design consisting of 16 runs, with the number of cube points (n_{c}) = 8, number of star points (nₛ) = 6, and combined number of center points in the cube portion of the design and the number of center points in the star portion of the design (n₀) = 2;
**α-L-arabinofuranosidase (AbfB) with volumetric activity of about 18.00 nkat mL⁻¹

### Preparation of Oatspelt Xylan Hydrogels for Assessing Effect of Plasticizer, Xylan Concentration and Enzyme Hydrolysis Duration on Size and Surface Charge of Enzymatically Produced Xylan Hydrogels

Oatspelt xylan solution (2.5 mL) and PEG1000 plasticizer of specified volume and concentration according to the CCD were mixed in a test tube to which 1.0 mL of AbfB (volumetric activity=18.0 nkat mL⁻¹) was added. The volume of the reaction mixture was adjusted to 5 mL with 0.1 M citrate buffer (pH5.0). The reaction mixture was incubated at 40° C. on a shaker with a rotating speed of 20 rpm.

The PEG-treated oatspelt xylan hydrogels were analysed for mean particle size and zeta potential using a Zetasizer (Nano ZS90) as previously described. The zeta potential of the AbfB formed xylan hydrogels was measured using a Zetasizer (Nano ZS90) operating based on a combination of Electrophoresis and Laser Doppler Velocimetry techniques (sometimes called Laser Doppler Electrophoresis) (Malvern Instruments, 2004). The method calculates the velocity of the particle in a liquid when an electrical field was applied. Given the particle velocity, the electrical field applied; viscosity and dielectric, the zeta potential can be calculated (Malvern Instruments, 2004). The results were analysed using an inbuilt Malvern software programme (Malvern Instruments, 2004). The morphology of the particles in the mixture was assessed by use of a microscope to which a camera (Nikon Ellipse E400) was attached at a magnification of ×600.

Encapsulation and Release of Horse Radish Peroxidase in AbfB formed Oatspelt Xylan Hydrogels

A mixture of oatspelt xylan and AbfB was prepared as described above in two sets of test tubes. In one set, 50 and 100 μL horse radish peroxidase (HRP Type VI-A P6782, EC 1.11.1.7) having a concentration of 1 mg mL⁻¹ and specific activity of 1000 U mg⁻¹ against 2,2′-azino-bis(3-ethylbenzthiazoline-6-sulfonic Acid) (ABTS) Sigma) was added. The reaction volumes were adjusted to 5 mL by adding 0.05 M citrate buffer (pH 5.0). The AbfB hydrolysis of the oatspelt xylan was performed either with or without HRP at 40° C. for 24 h. The total reaction volume in both sets was 5 mL. The reactions were stopped by placing the test tubes in water containing ice. After 1 h of terminating the reactions, 50 and 100 μL HRP was added to the second set. Subsequently, both sets were stored at 4° C. for at least 24 h prior to testing for HRP positive encapsulation. Two control samples (1) containing oatspelt xylan solution and AbfB in absence of HRP and (2) containing HRP and oatspelt xylan with HRP (HX) but without the AbfB were processed under the same incubation conditions. The set in which HRP was added during AbfB hydrolysis was labelled AHB and the one in which HRP was added after AbfB hydrolysis was labelled AHA.

Samples (2 mL) were taken from the xylan precipitates and centrifuged at 10 000 rpm for 5 min at 4° C. The supernatant was decanted to recover the pellet (hydrogel). The pellet was washed by re-suspending it in one volume Milli-Q H₂O and again centrifuged at 10 000 rpm for 5 min at 4° C. The process was repeated until there was no trace of HRP activity in the decanted water. The HRP activity in the decanted water was tested using a modified version of an assay protocol prescribed by Sigma. About 20 μL of the xylan hydrogel was placed in an eppendorf in which 100 μL of 9.1 mM ABTS (prepared in 100 mM potassium phosphate buffer pH 5.0) and 100 mM potassium phosphate buffer pH 5.0 in de-ionized water to make a total volume of 265 μL was added. About 5 μL H₂O₂ (0.3%) prepared from 30% (w/w) stock solution (Merck) was added to the mixture. Thereafter, the reaction mixture was incubated in the dark for 10 min. The assay was conducted at room temperature (25° C.).

The degree of encapsulation and rate of release of the HRP out of the AbfB formed oatspelt xylan hydrogel matrix were determined for HRP added before and after AbfB xylan hydrogel formation, denoted as AHB and AHA, respectively. The degree of encapsulation was calculated based on the optical density measured at 405 nm (OD₄₀₅ₙₘ), a microplate reader (xMark™ Bio-rad) against a reagent blank which contained ABTS, buffer and H₂O₂. The OD₄₀₅ₙₘ change due to oxidation of ATBS substrate by the HRP was read after 10, 30, 60, 120 and 180 min from the commencement of the assay. The samples remained in the dark during the measurement intervals. The rate of diffusion of the HRP out of the xylan hydrogel was defined as the rate of change of the OD (OD₄₀₅ₙₘ) per unit time (ΔOD min⁻¹). In the assays ATBS oxidized by HRP that was not encapsulated in the xylan hydrogels was used as a positive control whereas a mixture of ATBS- and AbfB-formed oatspelt xylan hydrogels in the absence of the HRP was used as a negative control.

### Results

### Production of AbfB

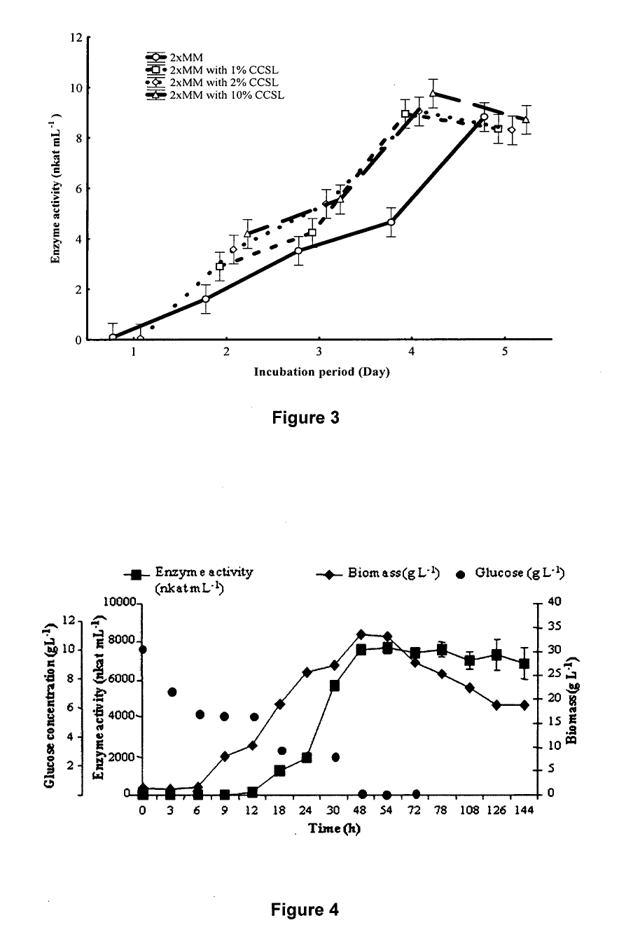

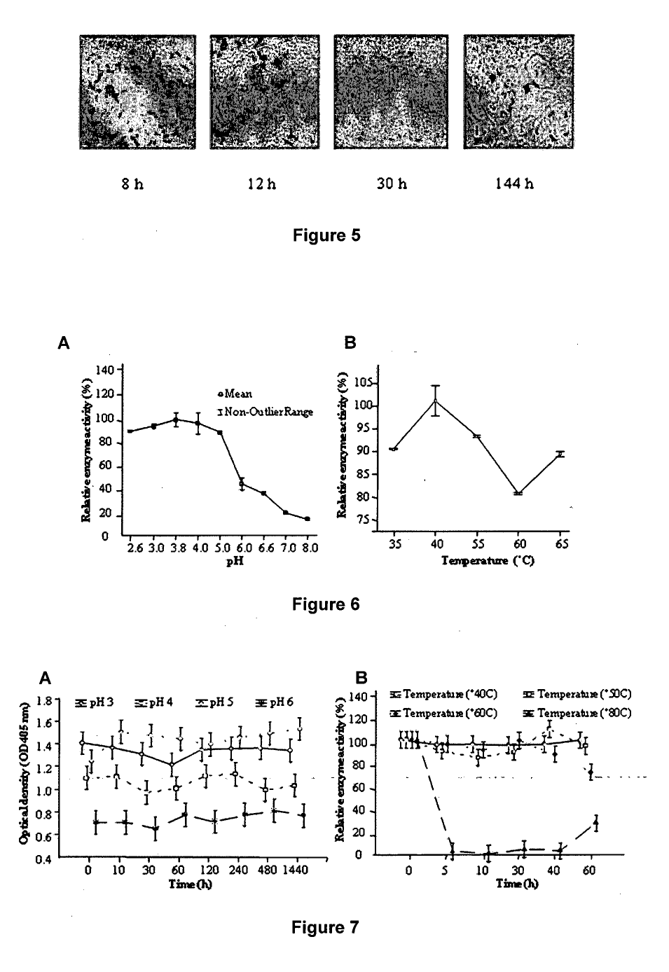

A. niger D15 was transformed with pGTP-abfB. The recombinant AbfB was expressed extracellulary in pellet and mycelial formation by A. niger cultivated in shake flasks and a bioreactor, respectively. The volumetric activity of the AbfB in the shake flasks reached a maximum of 10.0 nkat mL⁻¹ on the 6^{th} day of incubation (FIG. 2). The volumetric activities of AbfB in CCSL in 1%, 2%, and 10% CCSL enriched media were by the second day 1.8, 2.2 and 2.6 times the volumetric activity in 2×MM, respectively. The Abfb activity in 2×MM with 10% CCSL and 2% CCSL reached a maximum activity of 10.0 nkat mL⁻¹ a day earlier than in the 2×MM media (FIG. 3). Notably, the AbfB activity in 2×MM with 2% and 10% CCSL were not significantly different (p<0.05) during the incubation period. The AbfB produced in the bioreactor in 2×MM with 2% CCSL had a maximum volumetric activity of approximately 8.0 nkat mL⁻¹ which was achieved after 36 h of incubation (FIG. 4). In crude form, the specific activity of the recombinant AbfB was 18.0 nkat mg⁻¹ (Table 3). The AbfB production increased with biomass growth. The biomass growth corresponded with a decrease in glucose concentration (FIG. 4). The biomass concentration of the A. niger grown on 2×MM 2% CCSL enriched medium in the bioreactor reached 32 g L⁻¹ (FIG. 4). During the incubation period, the morphology of A. niger was observed to change from pellets to an extensive network of mycelia (FIG. 5). The mycelium showed signs of cell lysis after 144 h of incubation which corresponded to a fall in biomass concentration and depletion of glucose in the bioreactor (FIGS. 4 and 5).

TABLE 3
Comparison of specific activity of crude and partially purified α-L-
arabinofuranosidase (AbfB)

| | Specific | | [p- | | |
|---|---|---|---|---|---|
| | activity | Specific | NPA] | | |
| | (nkat | activity | in | | |
| Enzyme | mg⁻¹) | (U mg⁻¹) | mM | Purity | Reference |
| A. niger | 48 | 2.9 | 5 | Crude | This study |
| (AbfB) | | | | | |
| A. niger | 45.3 | 2.7 | 5 | Partially | This study |
| (AbfB) | | | | purified in | |
| | | | | ammonium | |
| | | | | sulphate | |
| A. niger B | 43.32 | 2.6 | 2.5 | Crude | De Ioannes |
| | | | | | et al., 2000 |
| A. niger | 2450 | 147 | 3.7 | Purified | Rombouts et |
| AraB | | | | | al. 1988 |
| A. terreus | 1880 | 113 | 2 | Purified | Luonteri et |
| Ara | | | | | al., 1998 |
| T. reesei Ara | 1760 | 106 | 2 | Purified | Luonteri et |
| | | | | | al., 1998 |
| A. nidulans | 383 | 23.5 | ns | Purified | Gielkens et |
| | | | | | al., 1999 |
| A. kawachii | 478 | 28.7 | 10 | Purified | Koseki et |
| AKAbf54 | | | | | al. 2006 |

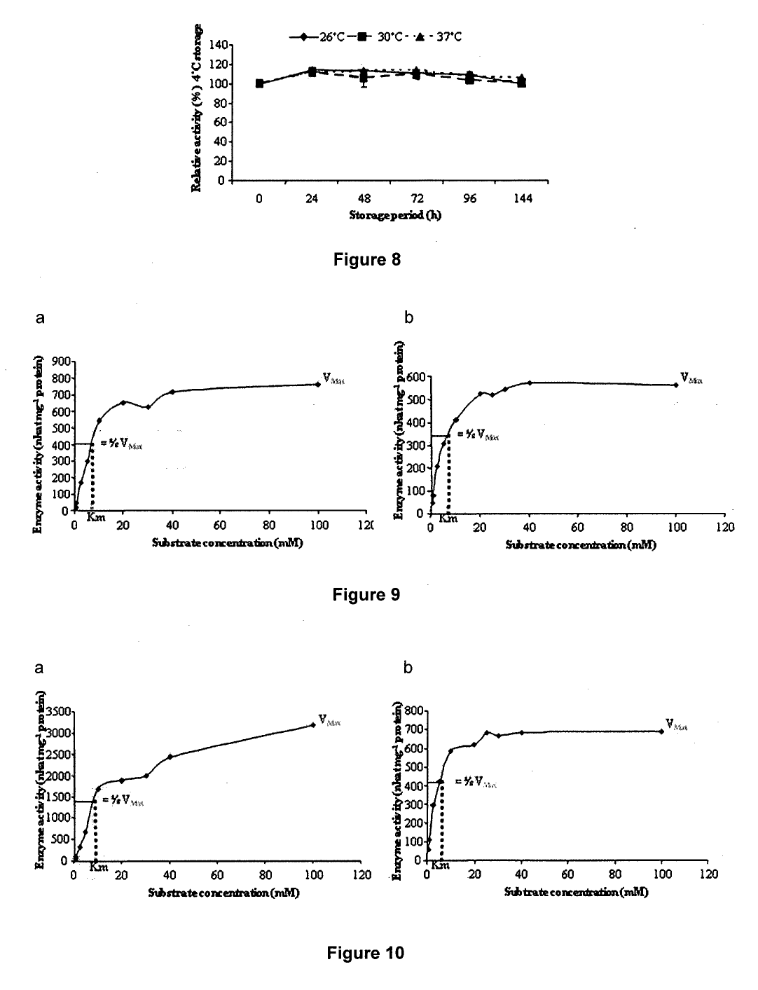

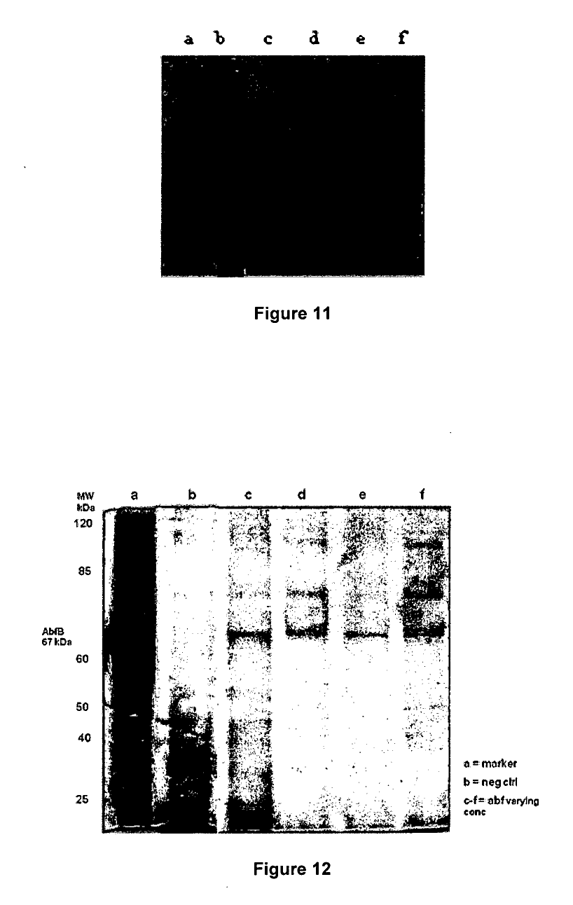

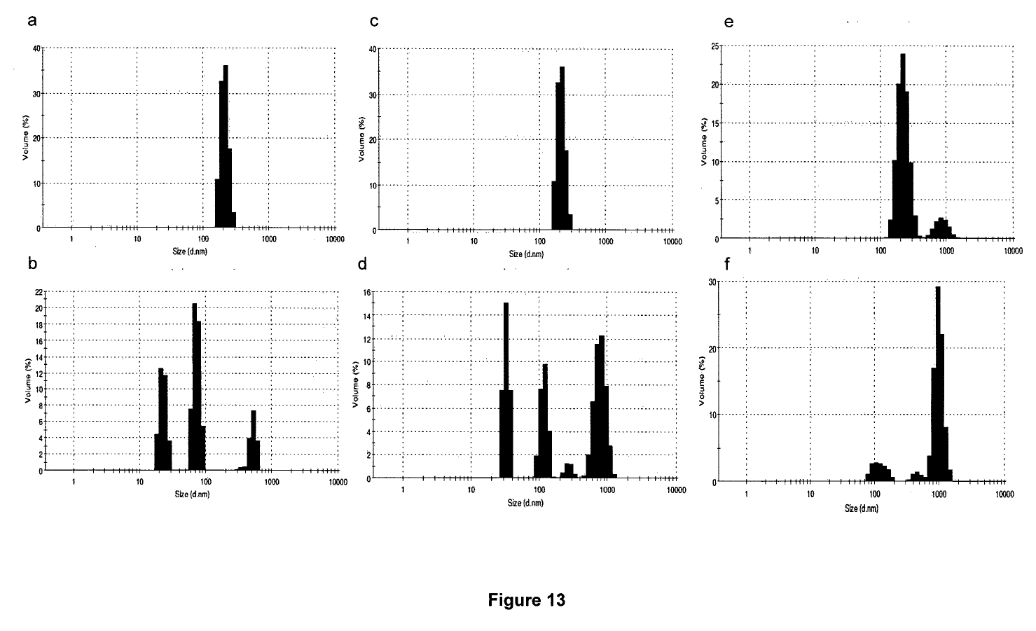

The optimal pH of AbfB ranged from pH 3.0 to pH 5.0 and was stable over pH 3.0 to pH 6.0 (FIGS. 6A and 7A). The recombinant AbfB displayed an optimal temperature of 40° C. and demonstrated stability at temperatures up to 60° C. for at least 30 min (FIGS. 6B and 7B). The AbfB was relatively more stable when incubated at 40° C. for 1 h but lost 95% of the activity within 5 min when incubated at 80° C. The relative residual activity against pNPA of AbfB, initially stored at 4° C., increased by 13% within 24 h of storage at 26, 30 and 37° C. (FIG. 8). The AbfB activity at the three storage temperatures remained stable up to 72 h and no significant differences (p<0.05) in the AbfB activities were observed for the entire storage period (FIG. 8). The dependency of crude and partially purified AbfB activity on pNPA concentration is presented in FIGS. 9 and 10. The initial specific activity against pNPA of crude AbfB obtained from bioreactor on 2×MM with 2% CCSL (w/v), from shake flasks on 2×MM with 2% CCSL (w/v) and on 2×MM medium increased linearly with increase in pNPA concentration, reaching a maximum velocity (Vₘₐₓ) at 40 rriM pNPA (FIGS. 9 and 10). The Km value (the amount of substrate to get the maximum velocity) was estimated at pNPA concentration of 10 mM. The velocity of partially purified AbfB by ÄKTA purifying process increased linearly with increase in pNPA concentration up to 10 mM and remained constant up to 40 mM. However, a second phase increase emerged beyond 40 mM. The molecular weight and isoelectric point (pI) of the AbfB enzyme estimated from abfB gene sequence using DNAMAN sequence analysis software were 52.5 kDa and pI 4.04, respectively, whereas the estimated molecular weight from silver stained (Biorad) 10% SDS-PAGE was 67 kDa (FIG. 11). Multiple protein species were visualised in the 10% SDS-PAGE of AbfB produced in the bioreactor using CCSL enriched medium and in the AKTA partially purified AbfB, whereas single protein species was present in the SDS-PAGE of the AbfB produced in shake flasks, both in 2×MM and CCSL enriched media (FIG. 12).

### Characterisation of AbfB Formed Oatspelt Xylan Hydrogel

AbfB hydrolysis of oatspelt xylan resulted in formation of hydrogel precipitates which aggregated with time. The aggregation of the oatspelt xylan hydrogel resulted in a two phase solid-liquid separation. The AbfB-formed xylan hydrogels appeared spherical when in dispersed form, whereas irregular shapes prevailed when in aggregated form. The AbfB-formed oatspelt xylan hydrogels were spherical and more dispersed at an oatspelt xylan concentration of 0.32% xylan than at a concentration of 3.68%.

In the absence of any plasticiser, the particle size range of the AbfB-formed oatspelt xylan hydrogels were estimated to be between 18 and 700 nm in diameter after 30 min of terminating the AbfB hydrolysis (FIG. 13b). Afterwards, the bulk of the hydrogels attained a mean particle size within the 100 nm range (FIG. 13b). However, after a 2.5 h interval (i.e. 3 h later), the particle size range of the AbfB-formed hydrogels was 28 nm and 1.28 μm (1280 nm), with the bulk of the xylan hydrogels attaining sizes of between 200 nm and 1 μm (FIG. 13d). After storage of the hydrogels for a period of 7 days at 4° C. the particle size range shifted further to be between 78 nm and 1.48 μm (FIG. 13f). The particle size of the control samples was initially between 150 and 200 nm (FIG. 13a), but after 7 days of storage the particle size ranged from 100 nm to 1 μm (1000 nm), with the bulk of the hydrogels attaining particle sizes of <1000 nm (FIG. 13e).

### Response Surface Analysis of the Effect of PEG1000 Plasticiser, Xylan Concentration, Enzyme Hydrolysis Time on Oatspelt Xylan Hydrogel Particle Size and Zeta Potential

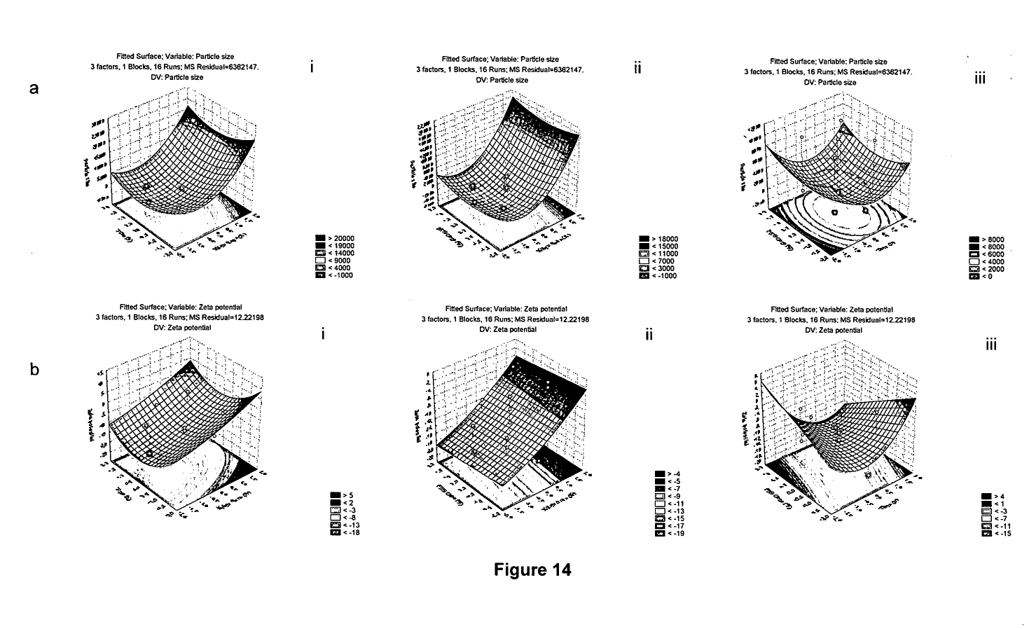

The response surface plots for the effects of xylan concentration, AbfB hydrolysis time and PEG 1000 concentration on particle size and zeta potential are shown in FIG. 14. At a constant AbfB hydrolysis time of 15.5 h and PEG1000 concentration of 27.7% the particles were at 3% predicted to be 20000 nm (20 μm) by the model (FIG. 14a i and ii). The response surface plot for the particle size fitted the polynomial quadratic equation with R² of 0.88 and adjusted R²=0.81 after ignoring interaction effects. The surface plot for zeta potential showed curvature with time but a linear relationship with PEG 1000 and xylan concentrations (FIG. 14b i-iii). The xylan hydrogels produced by the AbfB gave negative zeta potential values ranging from −19 to −1.03 mV (Table 2). At a xylan concentration of 1.5%, a zeta potential value of =−14.3 mV was estimated at PEG 1000 concentration of 27.5% with AbfB hydrolysis time held constant at 15.5 h (FIG. 14b ii) compared to −12.5 at 2% xylan concentration. The surface plot showed that at zero AbfB hydrolysis time (coded as −2), PEG1000 concentration of 27.5% and xylan concentration of 2%, the zeta potential was predicted as −2 mV, whereas at a PEG concentration of 27.5% and AbfB reaction time of 1 h the zeta potential was −3.32 mV (FIG. 14b iii). By increasing the AbfB hydrolysis time to 28 h and maintaining PEG 1000 concentration at 27.5%, a zeta potential value of −9.0 mV was obtained (FIG. 14b iii). The response surface plot for the zeta potential fitted the polynomial quadratic equation with R² of 0.74 and adjusted R²=0.56 after ignoring interaction effects involving xylan concentration.

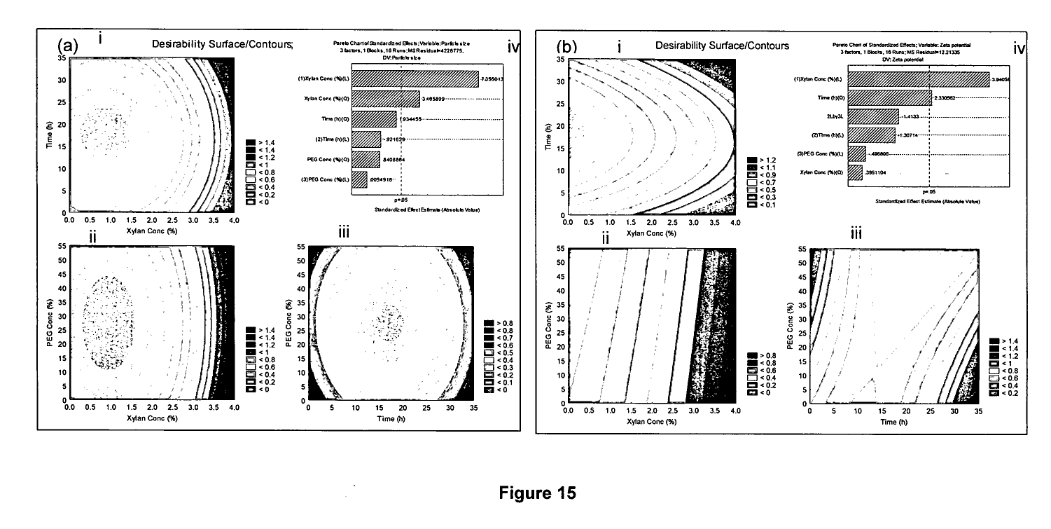

According to the desirability function, the AbfB formed hydrogels were the smallest at a xylan concentration of 1%, PEG concentration of 25% and hydrolysis time of about 17.5 h (FIG. 15a i-iii). The largest significant effect (p<0.05) observed on the particle sizes of oatspelt xylan hydrogels was the linear effect of xylan concentration, followed by its quadratic effect (FIG. 15a iv). The desirability function plot for zeta potential showed that the zeta potential values were more negative at low xylan concentration <1% (FIG. 15b i and ii) and when AbfB hydrolysis time was greater than 15 h (FIG. 15b iii). The largest significant effect (p<0.05) observed on zeta potential was the linear effect of xylan concentration, followed by quadratic effect of AbfB hydrolysis time (FIG. 15b iv).

### Encapsulation of Horse Radish Peroxidase in AbfB Formed Oatspelt Xylan Hydrogels

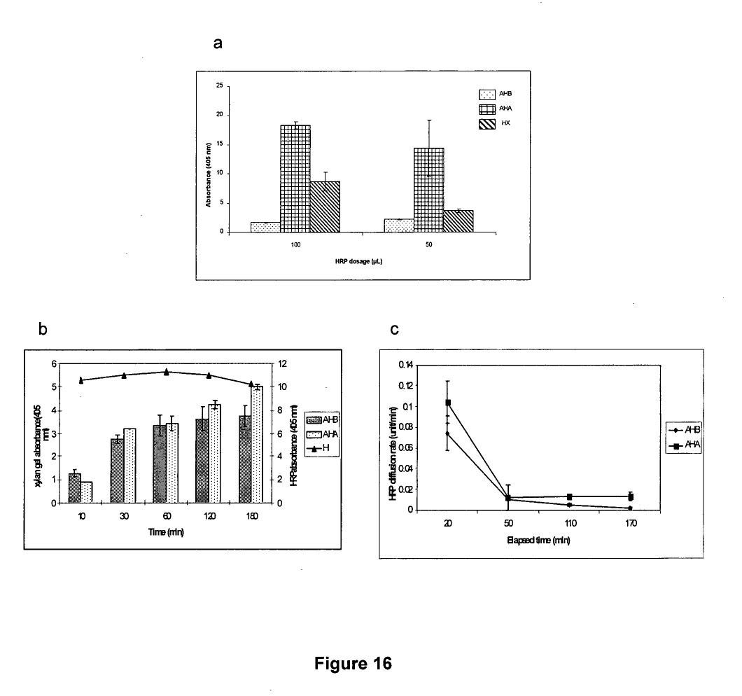

After thorough washing of the AbfB-formed xylan gels encapsulating the HRP with Milli-Q H₂O (thus washing done until HRP activity against ABTS substrate was no longer detectable in the waste water), the presence of the HRP was detected in the AbfB oatspelt xylan formed hydrogels as shown in FIG. 16. The residual activity as reflected by optical density (OD) at 405 nm of HRP in the oatspelt xylan reaction mixture was less for the HRP that was added before (AHB) than when added after (AHA) AbfB formation of the hydrogels (FIG. 16a). At a dosage level of 100 μL, the optical density of AHB in comparison to AHA and HX (HRP in untreated oatspelt xylan) was 1.0:11.0:5.1 (FIG. 16a). A similar trend was observed at a HRP dosage level of 50 μL in which the OD₄₀₅ₙₘ observed for AHB, AHA and HX was in a ratio of 1:6.5:1.7 (FIG. 16a).

Release Characteristics of HRP from AbfB Formed Oatspelt Xylan Hydrogels

The release characteristics of the HRP from the AbfB oatspelt xylan hydrogels were monitored by change of OD₄₀₅ₙₘ for a period of 180 min (3 h). The HRP activity in AHB and AHA hydrogels after 10 min of terminating the encapsulation process gave an OD₄₀₅ₙₘ of 1.276 and 0.882, respectively (FIG. 16b). The OD₄₀₅ₙₘ of HRP activity in AHB and AHA increased to 3.74 and 5.0 after 180 min, respectively. The OD₄₀₅ₙₘ of activity of unentrapped HRP monitored during the same period remained relatively steady at an OD₄₀₅ₙₘ of approximately 11 before dropping to 10 (corrected for dilution) after 180 min (FIG. 16b). The rate at which the HRP was released from the oatspelt xylan matrix (defined as the change in colour intensity (OD_{405 nm}) was in the initial 20 min 0.07 and 0.1 Umin⁻¹ for HRP in AHB and AHA, respectively (FIG. 16b). Over a 50 min time interval, the rate of release for AHB and AHA declined to 0.01 and 0.012 Umin⁻¹, respectively. Beyond the 50 min mark, the release rate of the encapsulated AHA stabilized at 0.013 Umin⁻¹, whereas that of AHB continued to decline by 50% for the subsequent measurement time intervals of 110 and 170 min (FIG. 16b).

### Discussion

### Characteristics of α-L-Arabinofuranosidase (AbfB) Produced Oatspelt Xylan Hydrogels

α-L-arabinofuranosidase was investigated as a tool for production of oatspelt xylan hydrogels that can be used as an encapsulation matrix and delivery system for bioactive agents. Commercially obtained oatspelt xylan was hydrolysed by α-L-arabinofuranosidase (AbfB). The AbfB reaction resulted in removal of the arabinose groups leading to formation of the xylan hydrogel precipitates. The oatspelt xylan hydrogels produced by the AbfB were spherically shaped and of nano sizes (FIG. 13). The results show that the AbfB has ability to induce production of xylan hydrogels with particle sizes of <100 nm in the absence of any additive. Therefore, use of AbfB for production of xylan nanohydrogels of spherical form simplifies the complex and costly methods described by other workers for preparing nanohydrogels from polysaccharides, such as precipitation, polyelectrolyte complexation and desolvation [Garcia et al., 2001, Polym. Bull. 46: 371-379; Wang et al., 2006, J. Mater. Chem. 16:3252-3267; Silva et al., 2007, Int. J. Pharm. 334:42-47; Daus and Heinze, 2009, Macromol. Biosci. 9 DOI: 10.1002/mabi.200900201]. The oatspelt xylan nanohydrogels produced by AbfB are of spherical form and enable uniform encapsulation and release of the bioactive substance.

The AbfB-formed xylan hydrogels with time formed aggregates that increased in size. The particle sizes of the oatspelt xylan hydrogels were between 18-700 nm after 30 min of terminating the hydrolysis reaction (FIG. 13b) and the hydrogels attained particle sizes with diameters between 28 nm and 1280 nm (1.28 μm) after 3 h (FIG. 13d). After seven days of storage at 4° C., the mean particle size ranged from 78 to 1480 nm (1.48 μm) (FIG. 13f). Despite the aggregation phenomenon, the lower particle size limit of the oatspelt xylan hydrogels produced by the AbfB of up to 78 nm was smaller than the lower particle size limit (162 nm) reported for nanoparticles produced from xylan by the esterification method [Daus and Heinze, 2009, Macromol. Biosci. 9 DOI: 10.1002/mabi.200900201], and produced from poly-(lactide co-glycolic acid) (PLGA) (380 nm) produced by a multiple emulsion method using dichloromethane (DCM). The findings suggest that oatspelt xylan hydrogels produced by the AbfB can be engineered to produce xylan hydrogels in a wide range of nanosizes suitable for specific administration regimes of bioactive agents.

### Effect of PEG1000 Plasticiser, Xylan Concentration, and AbfB Hydrolysis Time on Oatspelt Xylan Hydrogel Particle Size and Zeta Potential

AbfB reaction time and xylan concentration controlled the xylan hydrogel particle size and zeta potential. The size and surface charge are important properties affecting stability and functionality of the hydrogels. Therefore, optimising AbfB reaction time and xylan concentration is proposed to enable production of specifically sized stable xylan hydrogels for particular end uses. The xylan concentration showed a significant effect (p<0.05) on both particle size (FIG. 15a iv) and zeta potential (FIG. 15b iv). The correlation between AbfB hydrogel particle size and xylan concentration (FIG. 14a) was obtained with an adjusted R² of 0.81. At a constant PEG1000 concentration of 27.7% and AbfB reaction time of 15.5 h, spherical xylan hydrogels with a mean particle size of 292 nm were evident at a xylan concentration of 0.32%, compared to >10000 nm present at a xylan concentration of 3.68%. In addition, the AbfB hydrolysis of oatspelt xylan at a concentration of 3.68% resulted in production of hydrogels that had a greater tendency to aggregate than at a xylan concentration of 0.32%.

Both the xylan concentration and AbfB reaction time effects were significant with respect to the zeta potential (FIG. 15b iv). The results in Table 2 show that the zeta potential for the xylan was negative, which is characteristic of hydrogels from polysaccharides with bioadhesive properties. The zeta potential of the oatspelt xylan hydrogels produced by the AbfB increased with increased AbfB reaction time (FIG. 14b i-iii) and xylan concentration (FIG. 14a i-iii). For instance, at a xylan concentration of 0.32% and 3.68%, the zeta potential values were −15.5 and −9.5 mV, respectively (Table 2). Higher zeta potential values (thus, less negative zeta potential values) indicate reduced electrostatic repulsion forces on the surface of the hydrogels. The electrostatic repulsion forces are responsible for keeping the particles in separation hence, reducing the tendency of the hydrogels to aggregate. Zeta potential values of at least −30 to −20 mV imply formation of hydrogels with stable physical structure by electrostatic repulsion. In this study, the closest zeta potential for stability was −19 mV (Table 2). The aggregation behaviour of the oatspelt xylan hydrogel correlated to particle size and zeta potential. For instance, the otspelt xylan hydrogels that formed at 3.68% xylan had a zeta potential of −9.5 mV and particle size >10000 nm.

The results show that the significant relationship between AbfB reaction time and zeta potential was a quadratic one (FIG. 15b iv). At an AbfB reaction time of 1.2 h the zeta potential was −5.18 mV, whereas at a 15.5 h reaction time the zeta potential of up to −17.7 mV prevailed (Table 2). Accordingly, more stable and smaller particles were present after a reaction time of 15.5 h than at 1.2 h. The AbfB reaction beyond 17 h resulted in less negative zeta potential values giving relatively bigger particles than at the AbfB reaction time of 15.5 (FIG. 14b i-iii). The phenomenon could be linked to loss of electrostatic repulsion force due to aging of the hydrogels that promoted self aggregation (FIGS. 13a-f). In an industrial set up, xylan concentration is proposed to be moderated by progressive addition of the xylan and enzyme at a specified ratio and rate, which would provide adequate residence time for formation of the AbfB catalysed hydrogels with continuous harvesting of the AbfB xylan hydrogels at a rate corresponding to their formation, with the inclusion of a dispersion step such as sonication.

Addition of the PEG1000 plasticiser was less effective in stabilising the morphology of AbfB-produced oatspelt xylan nanohydrogels than oatspelt xylan concentration and hydrolysis time. The effect of PEG1000 plasticiser on particle size and zeta potential of the AbfB formed xylan nano hydrogels was studied in a central composite designed experiment (Table 2). The results showed that addition of the PEG1000 plasticiser was not significant on both the particle size (FIG. 15a iv) and the zeta potential (FIG. 15b iv).

### Horse Radish Encapsulation and Release

The AbfB-produced oatspelt xylan hydrogels encapsulated horse radish peroxidase (HRP) and released the HRP in active form. The encapsulation occurred both when the HRP was added before and after AbfB formation of oatspelt xylan hydrogel denoted as AHB and AHA, respectively (FIG. 16b). Successful encapsulation of the HRP by both procedures presents the AbfB as a flexible novel technology encapsulation and slow release of bioactive agents.

The AbfB-formed xylan hydrogels released active HRP over a period of 180 min (FIG. 16b). The sustained release of the HRP over the 180 min (FIG. 16c) indicates that the AbfB-formed xylan hydrogels can be used as an encapsulation matrix for slow release of bioactive substances. The release characteristics of the AbfB-formed xylan encapsulation hydrogels monitored over the 180 min showed a higher initial rate of release for the HRP encapsulated by both AHB and AHA methods (FIG. 16c). The initial higher rate of the HRP release is an indication of loosely encapsulated HRP. The results further imply that less than 100% encapsulation of the HRP had occurred. The observation was confirmed by the residual HRP activity evident in the reaction mixtures for both AHB and AHA encapsulation modes after removing the hydrogels (FIG. 16a). A balance between the rate of release and size of the hydrogels is proposed to increase the efficiency in the encapsulation and release of the bioactive substance.

Both AHB and AHA modes of encapsulation demonstrated slow release delivery mechanisms. However, there were differences in the release characteristics of the HRP between the AHB and AHA encapsulation modes. The initial rate of release of the HRP was almost 1.5 times higher in the AHA than in the AHB encapsulation method (FIG. 16c). After the initial one hour, the rate of release of the HRP from the AHA continued to be higher than from the AHB (FIG. 16c). The rate of HRP release from AHB encapsulation after the initial outburst continued to decline by 50% for each successive measurement interval. In contrast, the rate of release of the HRP from the AHA encapsulation stabilized after the initial outburst in the first hour (FIG. 16c). The phenomenon implies that the HRP was released with less restriction from the AHA encapsulation than from the AHB. Furthermore, such a difference is a reflection of the differences in the method of attachment of the HRP to the hydrogel. It is proposed that HRP encapsulated using the AHB mode was physically suspended inside the hydrogel matrix during the xylan hydrogel formation. Therefore the HRP release from AHB is proposed to be restricted with the thickness of the hydrogels and crosslinkages. The release characteristics of the HRP encapsulated by the AHA method, however, suggest the encapsulation occurs by adsorption onto the surface of the AbfB formed xylan hydrogel, allowing the HRP to be easily detached from the matrix. In addition, the release of the HRP that is adsorbed on the surface is proposed to be less affected by the changes in the morphology of the hydrogels than the HRP encapsulated inside the hydrogel because of new movement restrictions emerged in the diffusion pathway. Slower release of the HRP encapsulated by AHB encapsulation is expected than that encapsulated by AHA encapsulation over 180 min (FIG. 16c).

### Production of Hydrogels Using Birch Xylan and α-D-Glucuronidase

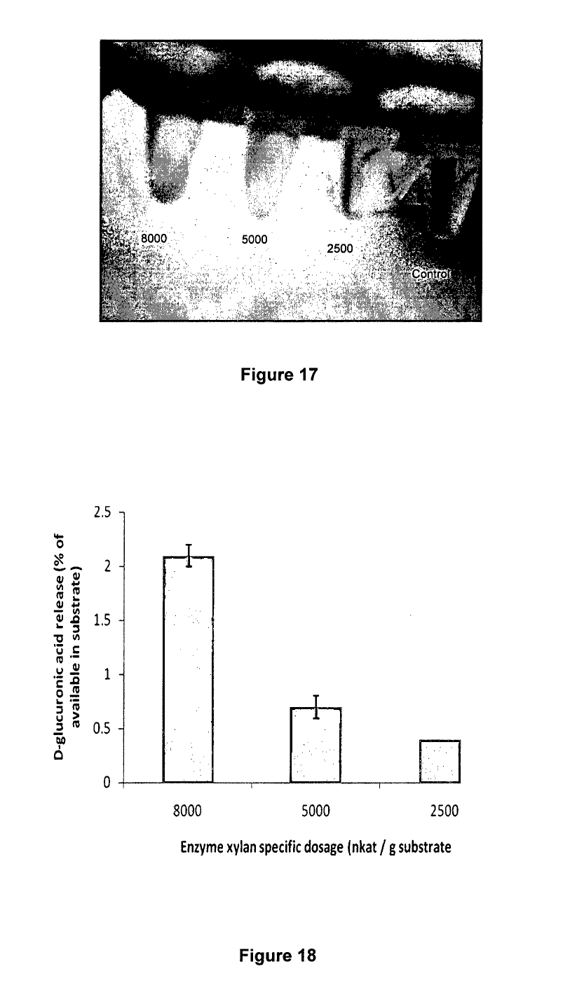

Using similar methods to those described above, the applicant also successfully formed hydrogels using α-D-glucuronidase to remove methyl-glucuronic acid side chains from birch xylan (FIGS. 17 and 18).

The hydrogel of the invention is useful for the production of nano and micro hydrogels from xylan, a biodegradable and non-toxic biopolymer source. This feature of the invention is desirable in the light of increased environmental and health concerns with regard to the use of petroleum based polymers.

The production of the hydrogel of the invention is simple, cost-effective, and can be precisely controlled without the use of toxic chemicals.

1. A method of producing a hydrogel, the method comprising the steps of:
enzymatically modifying xylan which contains glucuronic acid or a derivative thereof and/or arabinose side chains so that it has reduced solubility in water compared to naturally occurring xylan, by selectively removing glucuronic acid and/or arabinose side chains from the xylan with one or both of α-D-glucuronidase and α-L-arabinofuranosidase; and
allowing the modified xylan to form a hydrogel which encapsulates a bioactive substance, wherein the bioactive substance is brought into contact with the modified xylan either before the hydrogel forms or after the hydrogel forms.

2. A method according to claim 1, which further includes the steps of contacting the modified xylan with a bioactive substance and allowing the bioactive substance to be immobilized on or within the hydrogel.

3. A method according to claim 2, wherein the bioactive substance is added to the xylan before the hydrogel forms.

4. A method according to claim 2, wherein the bioactive substance is added to the xylan after formation of the hydrogel.

5. A method according to any one of claims 1 to 4, wherein the xylan is modified by selectively removing glucuronic acid side chains or derivatives thereof with α-D-glucuronidase.

6. A method according to any one of claims 1 to 4, wherein the wherein the xylan is modified by selectively removing arabinose side chains or derivatives thereof with α-L-arabinofuranosidase.

7. A method according to any one of claims 1 to 4, wherein the xylan is modified by selectively removing both glucuronic acid and arabinose side chains with α-D-glucuronidase and α-L-arabinofuranosidase.

8. A method according to any one of claims 1 to 7, wherein the xylan is modified by also selectively removing acetyl groups with acetyl xylan esterase.

9. A method according to any one of claims 1 to 8, wherein the source of the xylan is selected from sugarcane bagasse, bamboo, eucalyptus, pine, oatspelt and birch.

10. A method according to any one of claims 1 to 9, which further comprises the step of extracting the xylan from a lignocellulosic material prior to the modification of the xylan.

11. A method according to any one of claims 2 to 10, wherein the bioactive substance is selected from the group consisting of a protein, an enzyme, an antimicrobial agent, a bactericide and a pharmaceutical compound.

12. A method according to any one of claims 1 to 11, wherein the xylan is modified without the main xylan chain being damaged or degraded.

13. A method according to any one of claims 1 to 12, wherein the xylan is modified in the substantial absence of other hydrolytic enzymes.

14. A method according to claim 13, wherein the xylan is modified in the absence of xylanase.

15. A method according to any one of claims 1 to 14, wherein a sufficient number of glucuronic acid and/or arabinose side chains are removed from the xylan so as to reduce the water solubility of the modified xylan.

16. A method according to any one of claims 1 to 15, wherein the xylan is modified at a temperature of from about 30° C. to about 60° C.

17. A method according to any one of claims 1 to 16, wherein the xylan is modified at a temperature of from about 35° C. to about 45° C.

18. A method according to any one of claims 1 to 16, wherein the xylan is modified at a temperature of from about 40° C. to about 50° C.

19. A method according to any one of claims 1 to 18, wherein the xylan is contacted with the enzyme for between about 9 and about 72 hours.

20. A method according to any one of claims 1 to 19, wherein the xylan is contacted with the enzyme for between about 15 and about 28 hours.

21. A method according to any one of claims 1 to 20, wherein the xylan is modified at a pH of from about pH 4 to about pH 6.

22. A method according to any one of claims 1 to 21, wherein the xylan to enzyme ratio is about 5:2.

23. A method according to any one of claims 1 to 22, wherein the xylan loading is from about 12.5 mL.g⁻¹ to about 25 mL.g⁻¹.

24. A method according to any one of claims 1 to 4 and 6 to 23, wherein the α-L-arabinofuranosidase enzyme loading is about 2.5-5 mL.g⁻¹.

25. A method according to any one of claims 1 to 4 and 6 to 24, wherein the α-L-arabinofuranosidase has a volumetric activity of about 18 nKat mL⁻¹.

26. A method according to any one of claims 1 to 5 and 7 to 25, wherein the α-D-glucuronidase enzyme loading is about 0.2 mL.g⁻¹.

27. A method according to any one of claims 1 to 5 and 7 to 26, wherein the α-D-glucuronidase has a specific activity of about 300 nKat mg⁻¹.

28. A method according to any one of claims 1 to 27, wherein the concentration of xylan is from about 0.32% to about 3.68%.

29. A hydrogel formed by enzymatically modifying xylan which comprises glucuronic acid and/or arabinose side chains according to the method of any one of claims 1 to 28.

30. A hydrogel according to claim 29, which is suitable for pharmaceutical use.

31. A hydrogel according to either of claim 29 or 30, which further comprises a bioactive substance.

32. A hydrogel according to claim 31, wherein the bioactive substance is selected from the group consisting of a protein, an enzyme, an antimicrobial agent, a bactericide or a pharmaceutical compound.

33. A hydrogel according to any one of claims 29 to 32, which is insoluble in water.

34. A hydrogel according to any one of claims 29 to 33, which comprises particles of from about 18 nm to about 1.48 μm.

35. A hydrogel according to claim 34, wherein the average size of the particles is less than about 100 nm.

36. A hydrogel according to either of claim 34 or 35, wherein the particles are spherically-shaped.

37. A hydrogel according to any one of claims 34 to 36, wherein the particles have a surface charge (zeta potential) in the range of from about −19 to about −1.03 mV.

38. A hydrogel according to any one of claims 31 to 37, wherein the bioactive substance is encapsulated within the hydrogel.

39. A hydrogel according to any one of claims 31 to 37, wherein the bioactive substance is immobilized on the surface of the hydrogel.

40. A hydrogel according to any one of claims 29 to 39, which is for use in drug delivery.

41. A hydrogel according to claim 40, which is for use in sustained-release drug delivery.

42. A hydrogel according to claim 40, which is for use in targeted drug delivery.

43. A hydrogel according to any one of claims 29 to 42, which is for use as a medical dressing.

44. A hydrogel according to any one of claims 29 to 39, which is for use as a coating.

45. A hydrogel according to any one of claims 29 to 39, which is for use in a scaffold for tissue engineering.
